(19) United States
(12) Patent Application Publication
(10) Pub. No.: US 2023/0102975 A1
(43) Pub. Date: Mar. 30, 2023

(54) SYSTEM AND METHOD FOR ENABLEMENT OF SLEEP DISCOVERIES THROUGH CHALLENGES

(71) Applicant: KONINKLIJKE PHILIPS N.V., Eindhoven (NL)

(72) Inventors: Yash Parag MOKASHI, Pittsburgh, PA (US); Monica H BUSH, Murrysville, PA (US); Jeffrey SHEBETICH, Pittsburgh, PA (US); Mark Steven ALOIA, Boulder, CO (US); Rim HELAOUI, Eindhoven (NL)

(21) Appl. No.: 17/490,390

(22) Filed: Sep. 30, 2021

(51) Int. Cl. A61B 5/00; G16H 20/70

(52) U.S. Cl. CPC A61B 5/4815; A61B 5/7475; A61B 5/681; G16H 20/70

(57) ABSTRACT

A system and method create a framework for personalized sleep hygiene self-experiments to allow users to derive their own discoveries on how the self-experiment has affected their sleep.

### CROSS-REFERENCE TO PRIOR APPLICATIONS

This application claims the benefit of U.S. Provisional Application No. 63/121,353, filed on 4 Dec. 2020 and 63/166,303, filed Mar. 26, 2021. These applications are hereby incorporated by reference herein.

### BACKGROUND OF THE INVENTION

### 1. Field of the Invention

The present invention provides a system and method of creating a framework for personalized sleep hygiene self-experiments to allow users to derive their own discoveries on how the self-experiment has affected their sleep.

### 2. Description of the Related Art

Multiple factors can impact the quantity and quality of an individual's sleep. Poor sleep has been linked to reduced mental, emotional, and physical wellbeing, as well as daily functioning. Acute and chronic health conditions, work and family commitments, and various habits and activities that are developed over time as part of an individual's daily routine can all impact one's ability to get consistently good sleep quantity and quality. These habits, referred to as sleep hygiene behaviors, can be very difficult to change when they interfere with good quality sleep. Sleep hygiene behaviors include keeping consistent sleep/wake times, using caffeine appropriately, timing physical activity, controlling light exposure, avoiding excessive noise, and managing stress, among other things. All of these are factors that can impact an individual's sleep if not managed properly during an individual's day.

Currently, information on sleep hygiene can be found readily online, but the content is generic, with the same information and directions provided for everyone. Once these habits are established, changing behavior around them is complex. As anyone who has tried to change their behavior can attest, just identifying a behavior that needs to be changed is not enough. Individuals must be willing to engage in behavior change, persist when the change is challenging, and correlate actionable and measurable value to their efforts in order to adopt this new routine. Each person's journey in embracing and maintaining change is unique and requires personalized support and engagement over time, taking into account both when an individual is struggling, and when they are progressing well. Taking that first step in making a behavior change requires a personalized approach and encouragement on an individual level to enable personal discovery that will better enable adoption of a new sleep routine.

The idea of self-experimentation is not new in clinical or behavioral science. Self-experiments allow people to explore what changes lead to improved health and wellness, including improved sleep. In fact, most of us do it throughout our lives. Where we fall short, however, is that our efforts are often neither guided nor properly supported. Improvements to the quantity and quality of an individual's sleep thus would be desirable.

### SUMMARY OF THE INVENTION

Accordingly, it is an object of the present invention to provide an improved system and method for improving quantity and quality of sleep that overcome the shortcomings of conventional systems and methods for improving sleep. This object is achieved according to one embodiment of the present invention by providing an apparatus and method that allow individuals to exercise choice through self-experimentation for short periods of time to derive their own insights and value from the experiment, in a supportive and personalized framework.

That is, allowing individuals to exercise choice through self-experimentation for short periods of time to derive their own insights and value from the experiment, in a supportive and personalized framework, may encourage long-term adoption/adherence to make a lasting change. The invention thus advantageously provides a method of creating a framework for personalized, sleep behavior self-experiments to allow users to derive their own discoveries on how their behaviors affect their sleep.

The overall concept of sleep discovery through context-setting articles and self-experimentation was tested with twenty-three individuals. The response to this user testing was overwhelmingly positive, with an average rating for the concept at 8.29 out of 10 possible points. When asked about the value of this sleep discovery concept, users rated the value at 8.7 out of 10 points. The concept was also tested in a take-home test, with seventy-five users, where the users were asked to go through the entire sleep discovery experience. The experience entailed reading a relevant article and doing the sleep discovery challenges over a period of six days. The results from this user study were also overwhelmingly positive, with more than 65% of users giving a 4+ star rating to the value of the challenge in helping them discover something about their sleep, and more than 80% claimed that they were more aware of how their behaviors affected their sleep after the experience. Many also planned to adopt the new behavior long-term after experiencing the benefits through the challenge paradigm of the disclosed and claimed concept.

An all too common approach to behavior change is often lackluster and users are given a new or modified habit to try without follow up. While it is a difficult to track whether the user has actually performed the suggested routine, we have found three methods which improve the interest of the user to engage, track and support a successful implementation of the suggested behavior change.

A first aspect of engaging the consumer or patient or user is to support the innate interest of the consumer through short, concise articles that engage by educating the user. The user is able to browse various topics from which they then choose a topic that interests them. Once selected, relevant and concise articles that build the consumer's knowledge around a subject which interests them are provided. This knowledge or education provides a foundation for which behavior change is initiated. Once the user finished reading the article, an option is provided to further engage the user.

To further engage the user's interest, a second aspect of engaging the consumer or patient or user is to complete an “engagement loop”, which entails a repetition of the modified behavior for a user-defined period of time within a (clinically) defined range. Based on the user's tracked activity, coaching is then provided. Such coaching is intended to steer the user toward continuing successful implementation or encouraging the user to continue to try should they be unsuccessful at first.

To still further engage the user's interest, a third aspect of engaging the consumer or patient or user is to provide a conversation based upon Natural Language Processing (NLP) to identify the user's difficulties and facilitators to succeed, as well as their feelings during the engagement loop. Retrieving such information from the user's free text input via NLP allows the system and method of the disclosed and claimed concept to advantageously reflect the user's difficulties, facilitators, and feelings back to the user in a motivational way and convey the user the feeling of being understood without being judged.

Such reflections allow validating the user's feeling, when applicable, and offer an empathetic and supportive style to the conversation. Such interaction not only improves the user's experience and engagement with the solution, but also allows the tailoring of how the system and method enhance the user's confidence and guides the user toward successful implementation. Through this “engagement loop”, the improved system and method not only suggest behavior change but also support the behavior change in a fashion that is conducive to long-term motivation and success.

Accordingly, aspects of the disclosed and claimed concept are provided by an improved method of enabling a user to improve sleep by increasing at least one of a sleep quantity and a sleep quality of the user, that can be generally stated as including deploying an application with which the user interacts, presenting to the user a number of readable materials that include at least one of a number of written articles related to the improvement of sleep and a number of questions asked of the user related to the improvement of sleep, determining, based at least in part upon a number of inputs to the application by the user that are responsive to the number of readable material, at least one of a number of motivations of the user for wanting to improve sleep and a number of preferences of the user regarding presentation of information via the application related to the improvement of sleep, based at least in part upon at least a subset of the at least one of the number of motivations and the number of preferences, offering to the user a number of challenges related to sleep improvement, receiving from the user a selection input that is representative of an acceptance of a challenge from among the number of challenges, periodically determining, based upon a number of compliance inputs that comprise at least one of a number of objective inputs related to the user and a number of subjective inputs from the user, whether or not the user is complying with the challenge, based at least in part upon the periodically determining, periodically providing to the user a communication related to the challenge, periodically receiving from the user a responsive input that is responsive to the communication, based at least in part upon the responsive input, periodically providing to the user a further communication related to the challenge, and periodically receiving from the user a further input that is responsive to the further communication.

Other aspects of the disclosed and claimed concept are provided by an improved system structured and configured to enable a user to improve sleep by increasing at least one of a sleep quantity and a sleep quality of the user, the general nature of which can be stated as including a processor apparatus comprising a processor and a storage, an input apparatus structured to provide input signals to the processor apparatus, an output apparatus structured to receive output signals from the processor apparatus and to generate outputs, the storage having stored therein a number of routines that comprise an application with which the user interacts and which, when executed on the processor, cause the system to perform operations that can be generally stated as including deploying the application for interaction by the user, presenting to the user a number of readable materials that include at least one of a number of written articles related to the improvement of sleep and a number of questions asked of the user related to the improvement of sleep, determining, based at least in part upon a number of inputs to the application by the user that are responsive to the number of readable material, at least one of a number of motivations of the user for wanting to improve sleep and a number of preferences of the user regarding presentation of information via the application related to the improvement of sleep, based at least in part upon at least a subset of the at least one of the number of motivations and the number of preferences, offering to the user a number of challenges related to sleep improvement, receiving from the user a selection input that is representative of an acceptance of a challenge from among the number of challenges, periodically determining, based upon a number of compliance inputs that comprise at least one of a number of objective inputs related to the user and a number of subjective inputs from the user, whether or not the user is complying with the challenge, based at least in part upon the periodically determining, periodically providing to the user a communication related to the challenge, periodically receiving from the user a responsive input that is responsive to the communication, based at least in part upon the responsive input, periodically providing to the user a further communication related to the challenge, and periodically receiving from the user a further input that is responsive to the further communication.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a depiction of an improved system in accordance with an aspect of the disclosed and claimed concept;

FIG. 2 is a further depiction of the system of FIG. 1; and

FIG. 3 is a flow chart depicting certain aspects of an improved method in accordance with the disclosed and claimed concept.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the expression “a number of” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The disclosed and claimed system and method advantageously provide a holistic approach for enabling self-discoveries (i.e. a realization, deeper understanding, and/or self-education) related to sleep and how lifestyle choices affect sleep, particularly the individual's own sleep. This approach involved a framework for personalized sleep hygiene self-experiments, which include personalized engagement, reflection, and motivational interviewing, to allow users to derive their own discoveries on about their sleep.

The disclosed and claimed concept can be broken down in a number of objectives that correspond to aspect of the disclosed and claimed concept:
Understanding the user (Personalization of User Experience);
Context setting (Spark Curiosity);
Self-experience challenges (Self Discovery);
Ongoing engagement and support; and
Empathetic conversation style through supportive reflection and feedback.

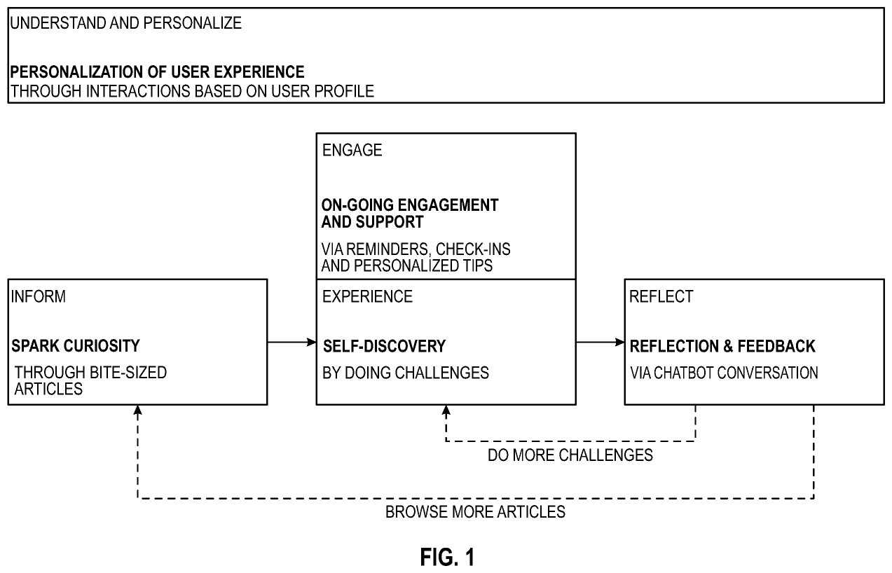

It is noted that these aspects of the disclosed and claimed concept do not necessarily occur in the same order as illustrated in FIG. 1 and are not intended to be construed as distinct steps. Rather, they are highly interrelated with one another. In a particular aspect of the disclosed and claimed concept, how each of these elements are implemented and contribute to a user's discovery around sleep and habits affecting sleep, is outlined in the following section and FIG. 1.

In FIG. 1, the “challenge” refers to the activity in which the user chooses to enroll and to commit to do for a defined/chosen duration, to experience how the habit affects their sleep. While recommended challenges are provided to users based on habits relevant to them and their preference, the choice to pick and commit to a challenge to up to the user.

A detailed description of the elements of the disclosed and claimed concept is set forth in greater detail.

### Understanding the User (Personalization of User Experience):

This section involves understanding the user's difficulties, preferences, objectives, and motivation for sleep improvement, and so that these issues can be utilized to display content and recommendations that are most relevant to the individual user. This helps provide a more personalized experience and provide information, features, and challenge parameters that are most relevant and important to the user to help in sleep discovery.

User Understanding can be Done Through a Combination of:
user responses to onboarding questionnaire and intermittent pop-up questions during the discovery journey;
user response to chatbot conversation in daily check-in conversations and challenge-end check-in conversation (automated conversation natural language processing (NLP) techniques), which can include detecting and storing (i) direct responses and information from the user, (ii) contextual sentiment and emotion detected from user's responses identified using NLP techniques of context-based emotion detection, and/or (iii) relevant semantic information such as their sleep issue, their reason to improve their sleep, and any particular topics or concepts related to sleep and the challenge;
tracking user interaction with an application 24 that is implemented on a mobile electronic device 28, for example through click tracking, timing of usage, geolocation during usage, bounce rate, drop-off points etc., including which specific articles and types of articles are read and/or browsed and the amount of time spent on each page, which challenges were opened/browsed, which application features were used/interacted with, etc.;
comparative machine learning techniques on similarity clusters, which can involve cluster analysis on anonymized data (which may include demographic data, objective data values, and other responses) related to all users in a user database to create cluster of similar users and to track historic motivations of similar users collected through a combination of subjective and objective input to predict the motivation of various users;
tracking user activities through activity data from a number of wearables 32, such as may include a biosticker or biosensor type devices when worn by a user that may detect parameters such as one or more of Heart Rate, Heart Rate Variability, Respiratory Rate, Activity, ECG, Skin Temperature, Body Position, Sleep staging, and/or SPO2, and wrist-worn consumer wearables (such as Fitbit, AppleWatch, Oura, etc.) may detect parameters such as one or more of Heart Rate, Heart Rate variability, SPO2, Respiratory Rate, Activity, Sleep Staging, Sleep Quality, Total Time in Bed, Sleep Onset Latency, and/or Skin temperature, by way of example and without limitation;
comparing user's input to the chatbot with the user's behavior and adherence patterns, which enables and allows to detect discrepancy between what the user wants to achieve and what the user actually is doing, it being noted that the developing of such a discrepancy through the chatbot can have an important effect on enhancing the motivation in selected behavior-triggered moments.

This ongoing process throughout the user experience builds user understanding as data are continually collected from the user through methods such as those mentioned elsewhere herein. The utilization of the user's understanding is seen in each element of the invention and will be highlighted in the corresponding sections.

The detected motivation and preference as referred to in this invention is an aggregate/combination of (i) the user's individual motivation and preference based on their objective data tracked through their interactions or sensors or through subjective questions and (ii) comparative ML techniques implemented on similarity clusters, driven by the assumption that users in the same cluster generated through multiple data points have similar motivations and preferences.

An important aspect about the user to be and feel “understood” is their “motivation for change” which is defined as why the user wants to improve their sleep, change their habits or derive discovery about sleep. Eliciting such information is enabled through a chatbot that leverages a set of rules to select the next action and response to the user based on their input. Such input can be a multiple choice or free text, by way of example, and relevant information, such as the user's reason to improve their sleep, is retrieved using NLP techniques as described above.

In particular, the user's reason for improving their sleep is used in two levels:
1) Content personalization: Through changing articles and challenge setup based on the user's motivation, i.e., more articles being displayed that are related to the user's motivation/goal, in context of habits and sleep. Similarly, the recommended challenges and challenge type are based on user motivation, for example challenges that are most likely to help user feel alert would be recommended if that is detected as the user's motivation.
2) Enhancing motivation level by reverting user's to their goals and strategically reminding them of their reasons to improve their sleep during the challenge, through check-in conversations and pop-ups. For example if a user wants to improve their sleep in order to feel more alert during university lectures, they are reminded of how doing the challenge activity will help them reach that goal. These interventions are particularly relevant when the user finds it difficult to stick to the challenge activity or when the user demonstrates reduced adherence and interaction with the application.

An understanding regarding the user's preferences advantageously helps to drive the customization and personalization of (a) the UI/UX (user interface and user experience) related to the application, (b) the challenges and articles, and (c) the type, style, content, and timing of interaction. User preference is determined through a combination of techniques listed above, including click/interaction tracking, user's subjective responses etc.

UI/UX preference is used to determine the kinds of interactions the user experiences in the application. This includes the type of interface, including styling and design, by way of example. This also includes the features that are displayed, the tone, style, and type of interactions in the conversations, check-ins, types of reflection, and other features that are set forth elsewhere herein. Multiple versions of the application with different combinations of UI/UX elements are generated through decision tree algorithms, and a new and/or customized versions of the application are deployed to the mobile electronic devices employed by the various users and are displayed to individual users based on their detected preference for each aspect of the UI/UX element.

The various challenges and articles which are displayed and recommended to the user are also customized based on user preference. For example, if the user preference and interest are known to be related to caffeine intake, then articles and challenges related to this topic are displayed. Difficulty and parameters of the challenge are also based on the detected preference.

While challenges and articles that are most relevant to the user are shown more often to the user, additional challenges and articles are also displayed on the application that might not be directed toward the detected preference. This enables the application to detect whether or not users are interested and motivated to change these other behaviors, along with educating users' other behaviors that might be affecting sleep.

User preference in relation of type of interaction and timing of interaction is used to customize the content, type, and style of messages displayed to the user, which is described in greater detail in the ongoing engagement section. The timing of these messages is also customized through the preference that is predicted of the user or the preference that is set by the user. Additionally, the interactions/ongoing engagement can be changed based on the momentary response of the user to them. For example, sentiment or mood that is detected at the time a message is received or the interaction that is displayed or through tracking of whether or not the messages are opened by the user.

### Context Setting (Spark Curiosity):

Before having the users enroll in self-discovery experiments and going through several days of exploration, this section helps set context for the discovery through reading content in the form of short articles. These articles provide information and aim to educate on how daily habits affect sleep. These are particularly relevant for habits that user is not aware about.

The selection of articles is based on the user's personal preference for type, content, style and length of content, and interest in particular topics. This user preference is derived from methods mentioned in detail in the “understanding the user” section herein. These articles are also based on predicted preference based on demographic information, viewing history, and goals compared with similar users using machine learning techniques of similarity clustering and dynamic persona generation. The articles link to additional reading resources, in the form of research papers, news articles or other digital media for users who are interested in learning more.

Each article has multiple tags associated with it. These tags relate to challenges, preference, motivation, and other aspect of user understanding derived from techniques listed in the “understanding the user” section herein. Articles are ranked based on the matching tags detected from the user. Articles that matches the user's preferences and motivations are displayed first. Each article has self-discovery challenges displayed on the same screen as the article. These challenges that are displayed are related to the content in the article. The choice of which articles to read and which related challenges in which to enroll is up to the user and is not enforced through the UX design.

### Self-Experience Challenges (Self Discovery):

After reading the articles, the user is offered a choice to enroll in the self-discovery challenges that are related to the article if they would like to learn more about the habits listed in the article and how they affect sleep. Each challenge is related to a particular habit. The challenge involves making a behavior change related to the habit that the user selects and commits to for the self-selected duration of the challenge. The enrollment of the challenge includes a description of how the habit is related to sleep, instructions for the habit change that is to be made for the challenge, customizable parameters related to the challenge, and customizable duration for which the user is committing to doing the challenge.

These habit changes are in a number of domain areas. Additional challenges are added for other lifestyle habits and aspects affecting sleep. The domain areas might include, for instance, caffeine intake, sleep schedule, stress reduction, exercise, etc., and the challenges are related to the domain areas. The initially displayed challenge with the recommended challenge parameters (which determine challenge difficulty) and duration are personalized to the user based on the user attributes and understanding of the user determined by techniques noted elsewhere herein. User can however change the parameter, difficulty, and duration of the challenge as they wish.

Dynamic challenge parameters are recommended based on the user's current baseline habit, such as a target caffeine reduction based on current caffeine consumption, etc. The system that determines these recommended challenge parameters, difficulty, and duration is a closed loop system based on performance of the user in past challenges, feedback from the user for other challenges, and also machine learning modeling based on performance and feedback from users in similar clusters. Recommendations based on comparison of similarity clusters are displayed to users in recommendations like “users like you liked this challenge”, “users like you picked this challenge”, “users like you generated discovery through this challenge”, etc.

Whether or not the user does the challenge activity is tracked daily via a number of compliance input that are obtained from the user, either through objective data or subjective data, depending on the user preference detected through methods described elsewhere herein. Objective data about the user may include sleep data (sleep duration, sleep efficiency, etc.), activity data (exercise timing, duration, active hours, etc.), light exposure, etc. through one or multiple connected wearables sensor devices like health watches, wearable devices, health tracking devices, and other devices such as are mentioned elsewhere herein. Subjective inputs from the user may include direct daily questions asked to user through daily check-ins, conversation, yes/no check boxes, aggregated data from multiple questions, detected emotion through NLP in responses, timing of responses, etc.

A user's success through challenges is tracked, and future challenges that are more suited for them are recommended. The additional challenges that are more suited can be recommended either while another challenge is still ongoing or at the end of the challenge.

A challenge is completed upon the elapse of the preset user-selected challenge duration. The user is provided a recommended challenge duration at the time of challenge enrollment, and user can customize this duration. Additionally or alternatively, the challenge duration can be customizable while the challenge is ongoing.

The motivation and tone of challenge are advantageously selected to encourage the user to at least simply try out the activities and are not necessarily related to success/failure of completion based on a target goal. However the challenges may additionally or alternatively be displayed in the UX in a fashion similar to that of a game, with the aim to complete the challenge successfully. The type of tone and UX displayed to an individual user is dependent on the user understanding and preference as detected through combination of methods listed elsewhere herein.

### Ongoing Engagement:

An advantage of digital health is its capacity for real time data collection and intervention, which overcomes several problems of in-clinic assessment and treatment (e.g., recall bias, not in-the-moment, etc.). Digital health applications are also becoming more popular as mobile electronic devices such as mobile phones and other devices are pervasive across all demographics. However, digital health solutions greatly depend on the quality of the collected data.

Previous work has demonstrated that the 30-day retention rate for using health and fitness apps is only 47% where the usage is 2.7 times a week on average. App Engagement: The Matrix Reloaded; http://flurrymobile.tumblr.com/post/113379517625/app-engagement-the-matrix-reloaded. Financial incentives are one way to engage participants. However, they may not be cost-effective to provide financial incentives for population-scale deployments. Engagement is closely related to motivation and persuasion. Fiske ST; Core social motivations; Handbook of motivation science; 2008: 3-22. Skinner's work on operant conditioning (Reynolds George Stanley; A primer of operant conditioning, Rev. 1975) and schedules of reinforcement (Ferster C B, Skinner B F; Schedules of reinforcement; Chicago: 1957) demonstrate how rewards and their frequency can shape behaviors. Cialdini showed how humans are influenced in a social setting. Cialdini RB; Influence; Vol. 3; A. Michel; 1987. BJ Fogg suggests that persuasion involves not only desirability outcome, but also having sufficient motivation, the ability to perform the behavior and being triggered to perform the behavior. Fogg BJ; A behavior model for persuasive design; Proceedings of the 4th International Conference on Persuasive Technology; ACM; 2009.

Thus, ongoing engagement with the user is a critical element of the challenge experience. At various points, the user is required to manually input relevant data associated with the challenge topic to inform next steps in coaching, i.e., number of cups of caffeine consumed, time of last intake of caffeine, etc. A common problem of manual logging is that users often disengage within a short time because of high burden.

As such, the self-experimentation challenges include engagement elements aimed at capturing scheduling, reinforcement by leveraging motivation, and persuasion for the user to engage with the challenge on a daily basis and encourage challenge completion. These elements to retain user engagement may include providing to the user communications such as:
daily reminders to check in to the challenge;
a reminder to complete the challenge;
reminders shown based on detected preference and customizable by user;
motivational messages to keep the user engaged with the challenge, including specific messages aimed when a user is showing success, i.e., a streak of completing the challenge activity for consecutive days, struggling with completing the challenge, i.e., not doing the challenge activity or disengaging with the application;
providing actionable tips based on a user's performance compared to other users or cluster of similar users generated through Machine Learning technique of similarity clustering; and
reminders and support to help complete the challenge criteria:
   timely reminders in the form of app or wearable device notifications based on activity, habits, schedule, similar clusters etc.,
   timing the reminders to when the user is most likely to take an action, and
   changing timing and/or type of interaction with user based on detected sentiment or “best time to interact”.

The application also receives from the user inputs that are responsive to communications from the application, such as those set forth immediately above, and such inputs from the user can be in the nature of, for example,
capturing their mood throughout the day, i.e., tracking discovery/reflection via personalization and timing of engagement based on sentiment/emotion/mood;
changing timing and/or type of interaction with user based on detected sentiment or “best time to interact”;
polling of users with associated questions on their challenge topic that can then be aggregated and provided back to other users with various metrics and information generated from analysis of users who fit a similar profile, i.e., “Did you know that 80% of people like you say they sleep 7 hours a night?”; and
at the beginning of the challenge, having the user select a personalized emotionally-based goal or outcome for the challenge that can be reflected back to them, especially if a user is struggling to complete a particular challenge, i.e., “Remember, sticking with this challenge will help you to ‘have fewer arguments with your wife because of your high stress’”.

### Reflection and Feedback:

Enabling the user to reflect on the challenge and challenge experience is an important aspect of enablement of discovery and deeper understanding, appreciation, and awareness about sleep and lifestyle habits affecting sleep. Reflection can be defined as the users themselves giving thought and consideration to how the challenge activity affected them and their sleep. This reflection is enabled through a number of features in the application.

One such features includes a check-in conversation chatbot which includes both voice and text chat interfaces. The conversation is in the nature of further communications between the application and the user and is delivered through chatbots, with conversation responses provided through decision tree algorithms. The conversation responses, i.e., output of the decision tree algorithm, from the application to the user may be in the form of direct questions, sentiment/emotion detected through NLP, and also other data points related to the user already stored in user profile database and collected through the methods described in the “understanding the user” section herein. As such, these further communications from the application to the user may be based on the user's responses to earlier communications in the conversation and/or other earlier communications. The further responsive communications received from the user in response to these further communications from the application are used by the application to further tailor additional and other communications to the user.

These check-in conversations can be anytime during the challenge, such as by giving the user a button option on the application interface to initiate a conversation, and/or at the end of the challenge duration, such as wherein the conversation is initiated when the user selects a notification provided at end of challenge duration. User can also initiate this end-conversation at a later time. Framing of the dialog is important as it provides a supportive environment that helps user think back on the discoveries.

While asking about the user's experience with the challenge, the conversation design is in the nature of empathetic responses which are incorporated in the chatbot responses to provide empathy when the user has a negative experience or a difficulty in completing the challenge. These are based on the intent and/or sentiment detected in user responses using NLP techniques and which drive the chatbot responses that are displayed to the user. This makes the user feel heard and acknowledges the difficulties the user faced while doing the challenge activity.

The chatbot conversation follows an empathetic style, and the flow is designed to steer the conversation to:
ask the user about their experience during, before, and/or after the challenge duration;
ask the user about their sleep, such as whether they noticed a difference in subjective sleep quality, alertness, and/or general wellbeing, while or while not doing the challenge activity or as an effect of doing the challenge activity;
ask the user to commit to incorporating the changes into their daily life if they saw a positive effect as a result of doing the challenge;
gain feedback on the challenge and experience related to the specific challenge to drive personalization on the type of challenges shown in the future and understanding of the user preference, and also to help improve challenges in the future; and
gain feedback during the challenge and make recommendations on changing the length of the challenge or other dynamic challenge parameters through analysis of user feedback and challenge data.

The tone, wording, and responses in the conversation are based on the percentage of completion, i.e., number of days they did the challenge activity or extent of challenge completion, thus providing the user with empathy and encouragement or celebratory congratulations. The tone of the conversation is personalized and modified based on the preference of the user as determined by the methods described elsewhere herein. Different conversation and changing challenge parameters may be based on detected sentiment and presence or absence of new discoveries related to the challenge habit affecting sleep.

While the degree of difficulty of the challenges may vary for different users, ongoing support is provided based on the detected performance, which is tracked through subjective input from the user and from objective data that may be tracked through wearables. Support to complete the challenge is provided in the form of actionable tips relevant to the particular challenge. These tips are added in the system in advance, for each challenge, and are annotated based on the clusters/user groups for which they worked best. Users are provided with these supportive tips based on predicted likelihood of benefiting the user. Example of tips would include suggesting to the user low caffeine or decaffeinated drink alternative if user is enrolled in caffeine reduction challenge. Other tips could be related to other behavior changes.

Reflection is further enabled via comparison of objective data by showing the user sleep metrics in graphical user interfaces, for example, over the duration of the challenge, and/or a comparison of sleep quality and metrics over days of doing and not doing the challenge activity. Alternately or additionally, the comparison could be highlighted in the form of encouraging pop-up messages or notifications depending on the detection comparison of sleep metrics. A report comparing sleep on days where challenge activity was done versus not done to highlight sleep improvement and relevance of the activity/habit in sleep improvement can also be provided. The type of interface and method of showing this comparison is determined based on user preference. Apart from sleep quality, objective data tracking of their motivation/goal is also done and displayed to the user as a correlation of improved sleep and the goal.

For instance, objective tracking of the improvement in running (or other athletic) performance can be provided when better performance is a motivation. By way of further example, objective facial recognition and showing a “score” for visual looks or alertness can be provided for those wanting to look fresher. By way of further example, tracking of weight can be performed if motivation is weight reduction. Other examples will be apparent.

Reflection is further enabled by developing discrepancy. On one hand, the system tracks the user's behavior through objective and subjective data, and on the other hand it elicits motivational input from the user such as their reason and goal from improving their sleep. These two might be at odds sometimes. If motivation drops over time, it important to detect that discrepancy and develop it through the chatbot feature to help the user elevate the importance of improving sleep in their life.

Reflection is also enabled via daily reflection on sleep quality. The user is daily during the duration of the challenge given an option to answer a question about sleep to make user reflect and think about their sleep and whether it is changing while doing the challenge activity. This supportive daily check-in includes an open-ended question asking about sleep and habit and a subjective question to get sleep rating for the previous night. These are followed up by reflections to validate and experience efforts and feelings and to affirm the user's efforts.

These methods can be implemented individually and/or in any combination and are deployed to the user application based on the detected user preference. This advantageously provides a personalized user experience to enable reflection and gain user feedback.

Furthermore, community-generated tips and sharing feedback enable reflecting to people based on what worked for other users in their cluster. This allow users to provide additional tips that worked for them in order to complete the challenge. Randomly selected user-generated tips can be provided to users with similar profile characteristics. If these are used successfully by users, they are incorporated into a master list of tips.

Other advantages can be provide by determining the most effective mix of engagement elements (i.e., articles, reminders, check in conversations, etc.) that were most successful for a particular consumer profile that successfully completed a challenge. These can be based on similarity clustering and their successful outcomes, and that series of engagement elements can be presented to other users that fit their consumer profile.

Still other advantages can be provided by an award and recognition system. This could be recognition in the form of an award or recognition system (example: badges or points) will be rewarded to users when they read articles, complete daily check ins, maintain streaks within a challenge, complete multiple challenges, complete check in conversations, etc. This can be done in order to encourage engagement and to build habits within the challenge structure. Recognition amounts will be weighted based on the significance of the action, with the most complex tasks, i.e., the completion of challenges or multiple challenges, being given the highest weight. Differentiation in reward amounts will also be driven by those tasks that are knowledge based (reading articles) versus supporting the user in changing their habits (challenges).

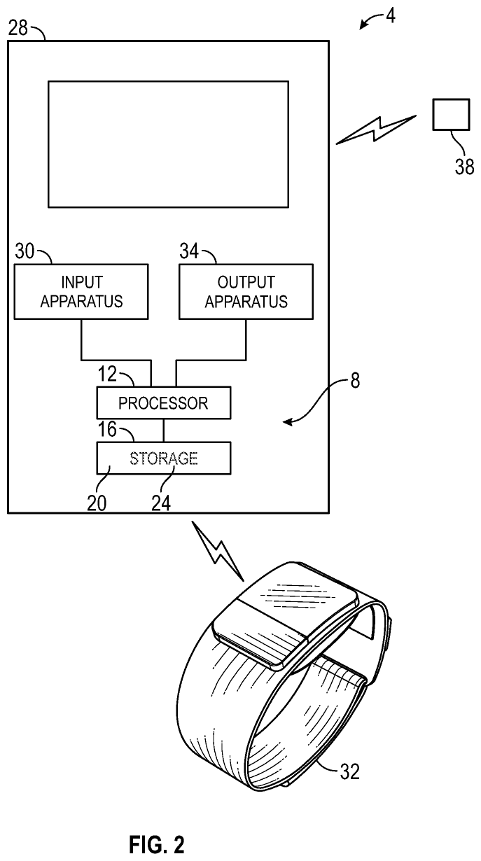

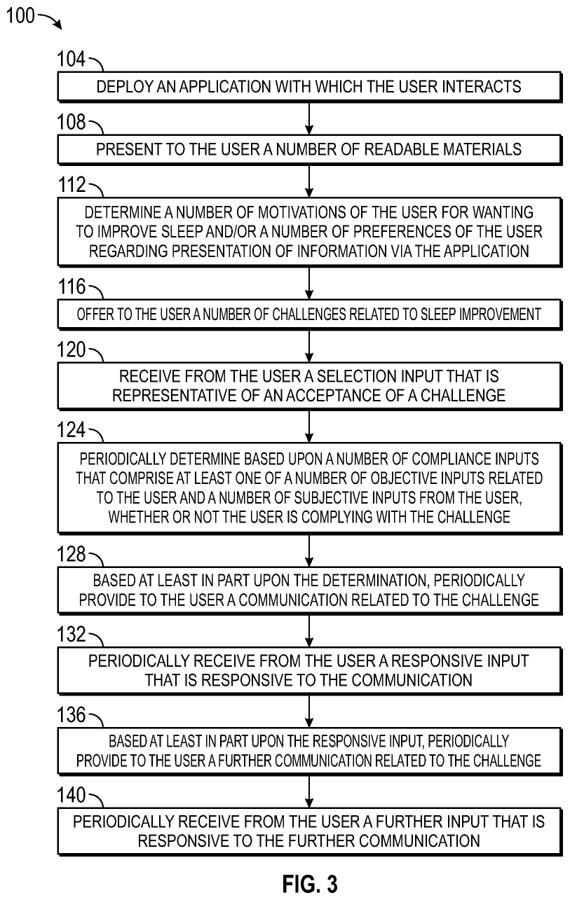

An improved apparatus 4 in accordance with the disclosed and claimed concept is depicted in FIGS. 1 and 2. Apparatus 4 can be employed in performing an improved method 100 that is likewise in accordance with the disclosed and claimed concept and at least a portion of which is depicted in a schematic fashion in FIG. 3. Apparatus 4 can be characterized as including a processor apparatus 8 that can be said to include a processor 12 and a storage 16 that are connected with one another. Storage 16 is in the form of a non-transitory storage medium that has stored therein a number of routines 20 that are likewise in the form of a non-transitory storage medium and that include the application 24 and other instructions which, when executed on processor 12, cause apparatus 4 to perform certain operations such as are mentioned elsewhere herein. Apparatus 4 further includes an input apparatus 30 that can include any of number of input components such as keys, input components of touchscreens, and a receiver component of a wireless transceiver, by way of example. Apparatus 4 further includes an output apparatus 34 that can include any of a wide variety of output devices that can include a visual display, a loudspeaker, and a transmitter component of a wireless transceiver, etc. Apparatus 4 can interface with an enterprise data system 38 and can further interface with the number of wearable 32, for example and without limitation. The enterprise data system 38 can deploy the application 24 to the mobile electronic device 28 or can execute it remotely and can compile data from other users that are similarly and otherwise situated compared with the user.

Certain aspects of the improved method 100 noted hereinbefore are depicted in the flow chart shown generally in FIG. 3. The method 100 can be generally stated to enable a user to improve sleep by increasing at least one of a sleep quantity and a sleep quality of the user. The method 100 includes, as at 104, deploying the application 24 with which the user interacts. The method 100 further includes, as at 108, presenting to the user a number of readable materials. Such readable materials can include at least one of a number of written articles related to the improvement of sleep and a number of questions asked of the user related to the improvement of sleep. Processing can continue, as at 112, with determining, based at least in part upon a number of inputs to the application 24 by the user that are responsive to the number of readable material, at least one of a number of motivations of the user for wanting to improve sleep and a number of preferences of the user regarding presentation of information via the application related to the improvement of sleep.

Based at least in part upon at least a subset of the at least one of the number of motivations and the number of preferences, processing can continue, as at 116, with offering to the user a number of challenges related to sleep improvement. Thereafter, processing continues, as at 120, with receiving from the user a selection input that is representative of an acceptance of a challenge from among the number of challenges.

Once the challenge has been accepted, as at 120, processing can continue, as at 124, with periodically determining, based upon a number of compliance inputs whether or not the user is complying with the challenge. The compliance inputs can comprise at least one of a number of objective inputs related to the user and a number of subjective inputs from the user. Based at least in part upon the periodically determining of compliance with the challenge at 124, processing can continue, as at 128, with periodically providing to the user a communication related to the challenge and, as at 132, periodically receiving from the user a responsive input that is responsive to the communication. Processing can continue, as at 136, wherein based at least in part upon the responsive input, the system 4 periodically provides to the user a further communication related to the challenge. Processing further continues, as at 140, with periodically receiving from the user a further input that is responsive to the further communication. Other benefits will be apparent.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method of enabling a user to improve sleep by increasing at least one of a sleep quantity and a sleep quality of the user, comprising:
deploying an application with which the user interacts;
presenting to the user a number of readable materials that include at least one of a number of written articles related to the improvement of sleep and a number of questions asked of the user related to the improvement of sleep;
determining, based at least in part upon a number of inputs to the application by the user that are responsive to the number of readable material, at least one of a number of motivations of the user for wanting to improve sleep and a number of preferences of the user regarding presentation of information via the application related to the improvement of sleep;
based at least in part upon at least a subset of the at least one of the number of motivations and the number of preferences, offering to the user a number of challenges related to sleep improvement;
receiving from the user a selection input that is representative of an acceptance of a challenge from among the number of challenges;
periodically determining, based upon a number of compliance inputs that comprise at least one of a number of objective inputs related to the user and a number of subjective inputs from the user, whether or not the user is complying with the challenge;
based at least in part upon the periodically determining, periodically providing to the user a communication related to the challenge;
periodically receiving from the user a responsive input that is responsive to the communication;
based at least in part upon the responsive input, periodically providing to the user a further communication related to the challenge; and
periodically receiving from the user a further input that is responsive to the further communication.

2. The method of claim 1, further comprising:
processing at least one of the input and the further input with a chatbot having a decision tree; and
employing a decision tree to generate at least one of the further communication and an additional communication.

3. The method of claim 1, further comprising periodically providing to the user as the communication a motivational communication related to the challenge.

4. The method of claim 3, further comprising providing to the user as the motivational communication related to the challenge a reminder to the user of the number of motivations.

5. The method of claim 1, further comprising:
offering at least one challenge from among the number of challenges that has a parameter that is customizable by the user; and
receiving from the user a customization input regarding the parameter.

6. The method of claim 5 wherein the customization input relates to at least one of duration and intensity of the at least one challenge.

7. The method of claim 5 wherein the customization input adjusts the parameter, and further comprising receiving from the user another customization input subsequent to the customization input that further adjusts the parameter.

8. The method of claim 1 wherein the communication is selected from among a plurality of alternative communications and is based at least in part upon a set of cluster data that is based at least in part upon a number of other users of the application.

9. A system structured and configured to enable a user to improve sleep by increasing at least one of a sleep quantity and a sleep quality of the user, comprising:
a processor apparatus comprising a processor and a storage;
an input apparatus structured to provide input signals to the processor apparatus;
an output apparatus structured to receive output signals from the processor apparatus and to generate outputs;
the storage having stored therein a number of routines that comprise an application with which the user interacts and which, when executed on the processor, cause the system to perform operations comprising:
deploying the application for interaction by the user;
presenting to the user a number of readable materials that include at least one of a number of written articles related to the improvement of sleep and a number of questions asked of the user related to the improvement of sleep;
determining, based at least in part upon a number of inputs to the application by the user that are responsive to the number of readable material, at least one of a number of motivations of the user for wanting to improve sleep and a number of preferences of the user regarding presentation of information via the application related to the improvement of sleep;
based at least in part upon at least a subset of the at least one of the number of motivations and the number of preferences, offering to the user a number of challenges related to sleep improvement;
receiving from the user a selection input that is representative of an acceptance of a challenge from among the number of challenges;
periodically determining, based upon a number of compliance inputs that comprise at least one of a number of objective inputs related to the user and a number of subjective inputs from the user, whether or not the user is complying with the challenge;
based at least in part upon the periodically determining, periodically providing to the user a communication related to the challenge;
periodically receiving from the user a responsive input that is responsive to the communication;
based at least in part upon the responsive input, periodically providing to the user a further communication related to the challenge; and
periodically receiving from the user a further input that is responsive to the further communication.

10. The system of claim 9 wherein the operations further comprise:
processing at least one of the input and the further input with a chatbot having a decision tree; and
employing a decision tree to generate at least one of the further communication and an additional communication.

11. The system of claim 9 wherein the operations further comprise periodically providing to the user as the communication a motivational communication related to the challenge.

12. The system of claim 11 wherein the operations further comprise providing to the user as the motivational communication related to the challenge a reminder to the user of the number of motivations.

13. The system of claim 9 wherein the operations further comprise:
offering at least one challenge from among the number of challenges that has a parameter that is customizable by the user; and
receiving from the user a customization input regarding the parameter.

14. The system of claim 13 wherein the customization input relates to at least one of duration and intensity of the at least one challenge.

15. The system of claim 13 wherein the customization input adjusts the parameter, and further comprising receiving from the user another customization input subsequent to the customization input that further adjusts the parameter.

16. The system of claim 9 wherein the communication is selected from among a plurality of alternative communications and is based at least in part upon a set of cluster data that is based at least in part upon a number of other users of the application.
